(19) United States
(12) Patent Application Publication
(10) Pub. No.: US 2024/0325375 A1
(43) Pub. Date: Oct. 3, 2024

(54) Effect of YEL002 on Psychological and Psychiatric Factors in Health and Disease

(71) Applicant: RadMit, Inc., Lake Balboa, CA (US)

(72) Inventor: Robert H. Schiestl, Los Angeles, CA (US)

(21) Appl. No.: 18/194,634

(22) Filed: Apr. 1, 2023

(51) Int. Cl. A61K 31/4709; A61K 9/107; A61K 47/44; A61P 25/00

(52) U.S. Cl. CPC A61K 31/4709; A61K 9/107; A61K 47/44; A61P 25/00

(57) ABSTRACT

A preparation comprising the YEL002 compound as an active ingredient for the prevention or treatment of one or more diseases, and methods of using such preparations. The preparations may be a sublingual pill, an injection, dissolved in liquid, dissolved in oil, or any other preparation. The conditions or diseases indicated may be psychological or psychiatric problems including anxiety/depression, aggression, alcoholism, drug and other addiction, insomnia, Alzheimer's and other dementia diseases, autism, schizophrenia, ADHD, PTSD, movement disorders and such.

### CROSS-REFERENCE TO RELATED APPLICATIONS

None

### BACKGROUND OF THE INVENTION

A previous application characterizing effects of YEL002 on diseases is disclosed in U.S. application Ser. No. 18/045,152 titled “Effect of YEL002 on Conditions and Diseases” and filed Oct. 8, 2022, of which the entire contents thereof are hereby incorporated by reference into the present disclosure.

Mental energy. Mental energy may be understood as the ability or willingness to engage in cognitive work. It is distinct from physical energy, and has mood, cognition, and motivation domains. Concepts closely related to mental energy include vigor and fatigue. An improvement of mental energy is very important for the psyche.

Sleep quality. Sleep quality is defined as an individual's self-satisfaction with all aspects of the sleep experience. Sleep quality has four attributes: sleep efficiency, sleep latency, sleep duration, and wake after sleep onset. An improvement of sleep quality is very important for health and psyche.

Anger and Aggression. A strong feeling that makes you want to hurt someone or be unpleasant because of something unfair or unkind that has happened. Since most killings are caused by aggression a treatment reducing aggression should also reduce the killing rate.

Furthermore, aggression plays a major role in the initiation and duration of war. So, the likelihood of war should also decrease when people are less aggressive.

The cause of arguments and fights is a lack of mutual, empathic understanding. When empathy is not engaged, then people revert to a self-protective mode and become judgmental. The result is a bad feeling on both sides and no happy ending.

Memory. The faculty of the mind by which data or information is encoded, stored, and retrieved when needed. It is the retention of information over time. Here are several common factors that can affect memory: Lack of sleep. Stress and anxiety. Depression. Memory is highly correlated with IQ and in any case, memory is 4-5 times better correlated with good grades in school and college than IQ.

Addiction. Addiction is defined as a chronic, relapsing disorder characterized by compulsive drug seeking and use despite adverse consequences. It is considered a brain disorder, because it involves functional changes to brain circuits involved in reward, stress, and self-control. There is evidence that addictive behaviors share key neurobiological features: They intensely involve brain pathways of reward and reinforcement, which involve the neurotransmitter dopamine. And, in keeping with other highly motivated states, they lead to the pruning of synapses in the prefrontal cortex, home of the brain's highest functions, so that attention is highly focused on cues related to the target substance or activity. It is important to know that such brain changes are reversible after the substance use or behavior is discontinued and treated.

Most people associate addiction with tobacco, drugs, and alcohol. A non-substance addiction includes things such as gambling, food, the internet, mobile devices, and shopping. These are sometimes called behavioral addictions. A treatment of addition should get rid of alcoholism, drug addition, smoking and all the other addictions.

Depression. Persistent sad, anxious, or “empty” mood, feelings of hopelessness, or pessimism, feelings of irritability, frustration, or restlessness, feelings of guilt, worthlessness, or helplessness, loss of interest or pleasure in hobbies and activities, decreased energy, fatigue, or feeling “slowed down”, difficulty concentrating, remembering, or making decisions, difficulty sleeping, early morning awakening, or oversleeping, changes in appetite or unplanned weight changes, thoughts of death or suicide, or suicide attempts, aches or pains, headaches, cramps, or digestive problems without a clear physical cause that do not ease even with treatment, suicide attempts or thoughts of death or suicide. Since 50% in young people and 80% in older people of suicides are caused by depression, a treatment of depression should reduce suicide rates.

Depression also causes: obesity, heart disease, Alzheimer's disease, diabetes, gastrointestinal problems, and asthma. Thus, a treatment of depression may result in a decrease of the prevalence of such diseases. Furthermore, about 50% of Alzheimer's as well as Parkinson's patients suffer from depression, which should reduce their disease burdens in addition to possibly treating these diseases as well.

Alzheimer's disease is the most common type of dementia. It is a progressive disease beginning with mild memory loss and possibly leading to loss of the ability to carry on a conversation and respond to the environment. Alzheimer's disease involves parts of the brain that control thought, memory, and language. A treatment for Alzheimer's should very much benefit this and maybe even other forms of dementia.

Costs of conditions. The worldwide prevalence and societal damage as it relates to a market size per year of these conditions and are as follows: Mental energy, treatment $100 billion; Sleep quality, treatment $110 billion; Mood enhancement, treatment $10 billion; Memory enhancement, treatment $6 billion; Addiction, worldwide 500 million people affected, treatment $700 billion per year; Anxiety and Depression, worldwide 800 million patients, worldwide societal loss per year $1 trillion, treatment $350 billion per year; Alzheimer's disease worldwide 800 million patients affected, treatment $300 billion per year.

### YEL002 Effects

Case studies show that COVID disease was treated and the Covid virus was cleared faster than without YEL002 and it supports the immune system to fight the virus and may also reduce the risk of infection. It may cure inflammation as well as autoimmune diseases caused by the virus and maybe even infections. There are 16 million new cases of COVID worldwide each week and 600 million cases of late COVID in the world which are due to continued inflammation and autoimmune diseases which also should be treatable.

A case study where it was used for eight weeks reduced symptoms of coughing and diarrhea, reducing the needs for drugs to treat those symptoms. Reportedly the compound was able to straighten and strengthen teeth such that the subject does not catch much food in their teeth any longer. It has also beneficial effects on the skin and one needs less of a cosmetic cream containing the same compound for the same effect.

In a user study, the patient's periodontics or receding gums were healed and no longer needed to floss teeth as much, since the teeth are now perfectly aligned and close together without a space in between. The patient also observed no longer needing eyeglasses after 55 years of wearing them, and after 4 weeks with the drug, the subject can see much better than anybody his age. According to the patient, the compound fixed 2 diopters and 1 astigmatism of eye-sight deficiency in only 4 weeks.

According to patient testimonial, his arms and legs are tighter and nurses have problems finding a vein for the first time in his life, since the veins moved further inside the extremities and are not on the surface any longer, thus it fixes varicose and spider veins. His blood is thinner, since proinflammatory factors make platelets sticky and cause blood clots. Thus, it prevents or treats blood clots.

According to another patient testimonial, one subject had twice elevated levels of liver damage ever since she had a gallbladder operation 25 years ago. After only two days of YEL002 treatment the values were normal and below the cutoff for the first time in 25 years as was her cholesterol.

According to another patient testimonial, one subject on chemotherapy was repeatedly vomiting many times a day, had no appetite, continually lost weight, did not eat, and did not want to live any longer. One week on YEL002 vomiting completely stopped, he had lots of appetite and is eating, gained 2 kg, and drove 320 km with his motorbike. He is extremely happy and all his doctors are happy too and told him that he needs to continue his experimental therapy.

According to another patient testimonial, one subject post-chemotherapy had coughing and wheezing symptoms and went downhill very fast and her daughter was worried that she was dying. After only one hour after YEL002 treatment this was completely alleviated and she slept well that night.

According to another patient testimonial, one subject was a heavy smoker and she had all kinds of inflammation problems including smoker's legs which were oozing lymph liquid and could not heal for many years. One week after YEL002 treatment that condition was completely healed and for the first time in years and her brush did not have any hair, meaning her hair loss completely stopped.

According to another patient testimonial, one subject dissolved YEL002 in oil and used that oil to spray her nose 2-4 times a day. This felt great and completely prevented all allergic attacks such as sneezing and runny nose.

Since diabetics have the same problem with their feet this could be fixed too, it is called peripheral arterial disease, so that nobody has to amputate feet or legs any longer, but just use YEL002 instead.

Based on the testimonials, it is apparent that the compound may be efficacious to improve lots of normal conditions and to benefit lots of diseases.

### BRIEF SUMMARY OF THE INVENTION

Disclosed are preparations comprising the YEL002 compound as an active ingredient for the prevention or treatment of one or more diseases. The preparations may be a sublingual pill, an injection, dissolved in liquid, or dissolved in oil.

Methods of preventing or treating diseases using a preparation as recited above are disclosed. Additional methods include applying an oil to the nasal membrane or atomizing the oil and inhaling the mist.

### DETAILED DESCRIPTION

The compound YEL002 has a myriad of medicinal and other therapeutic properties, some of which were previously described in U.S. Pat. No. 9,045,474 titled “Compounds and compositions for mitigating tissue damage and lethality” and in U.S. application Ser. No. 18/045,152 titled “Effect of YEL002 on Conditions and Diseases.” The patent discloses embodiments for various formulations containing YEL002. Embodiments as to use, dosage, and effects are described herein but all uses are included and not limited by the descriptions.

The molecular structure of the YEL002 compound is described below:

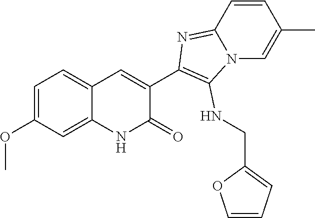 [SMILES: COc1ccc2cc(-c3nc4ccc(C)cn4c3NCc3ccco3)c(=O)[nH]c2c1]

The dose to use is about 1 gram per day for a 50 kg person which is about 20 mg/kg, the same as was used in previous experiments with mice.

The compound takes the appearance of an off-white powder and is fat soluble. One takes an eighth teaspoon or about 0.5 g dissolved in a couple of teaspoons of oil. In an exemplary embodiment, it is dissolved in grape seed or any other appropriate oil and taken twice a day, once in the morning and once in the evening. It is easy to use and has almost no taste.

The compound can also be delivered into the bloodstream through the nose as a nose spray. The compound is dissolved in oil and sprayed into or applied inside the nose. Furthermore, YEL002 can be applied just by itself or during inhalation of a mist produced by atomizing the oil and inhaled. All other delivery methods, such as the powder by itself, in pills, preferably sublingual pills, transdermal, or as injections, are also contemplated.

YEL002 has blood-thinning properties. Thus, if nose bleeding occurs at that dose when blowing one's nose, one should reduce the dose to half and nose bleeding will stop within about 12 hours or less. Once the flue or sneezing is gone one may increase the dose again.

YEL002 has a huge number of uses and holds promise as a most useful future pharmaceutical. In a high-throughput cellular screen at UCLA for a radiation mitigator funded by the National Institute of Health with $31 million during 10 years, 16,000 compounds from different chemical libraries were screened.

There were 10 primary hits and 6 confirmed hits and 2 of them were used in mice. Mice were irradiated with an LD100 after which 100% of the mice died after 10 to 15 days. When the researchers injected YEL002 IP into the irradiated mice 24 hours after radiation and another four doses in 24 hours intervals 100% of the mice, so all mice which would otherwise have died survived which was a highly significant difference. Many lived a normal life span of more than 2 years which is interesting since other labs which isolated other compounds, their mice died of late radiation effects much earlier after initially surviving.

It is speculated that this is because the primary screen applied was against radiation-induced toxicity as well as genotoxicity which causes cancer. Researchers involved in other labs were screening only against radiation-induced toxicity and thereby cells with genotoxicity survive and cause even more cancer and late effects of which surviving mice died. In our experiments, however, a screen against radiation induced toxicity as well as genetic instability made sure that the surviving cells also had no genetic instability which otherwise would have led to an increase in cancer and other diseases. In the experiment, ATM-deficient mice injected every week with YEL002 during most of their lifespans lived 33% longer than without injections.

In another experiment at the National Institute of Health, in other mice after radiation exposure which caused 90% of them to develop lymphoma, there was a reduction of the lymphoma incidence to 40% and the spontaneous frequency was reduced from 10-20% down to 0%. The compound was shown to work in yeast, human tissue culture cells, zebrafish and mice. Extrapolating these results to the life expectancy of humans, it is speculated that humans could live 20 years longer and with a reduction of cancer and probably many degenerative diseases as well as cancer. This was observed after treating mice right after birth, if humans are treated later in life it is possible that only 10 or 5 years are added and probably more for DNA repair deficient diseases like ataxia telangiectasia (ATM) which is still very substantial, especially, since it treats and reduces the likelihood of many diseases.

The compound made cells more resistant to exposure to a variety of agents, such as UV, chemical carcinogens, chemical warfare agents, radioactive iodine and cigarette smoke.

In case of radiation accidents or nuclear reactor accidents such as Fukushima or Chernobyl, or exposure to a nuclear or a dirty bomb, for the army against chemical warfare agents like in the Iran-Iraq war, for stewardesses, pilots and astronauts which have a higher occupational risk of cancer and frequent fliers or any other flier against space radiation effects, against radon in the basement induced lung cancer, against exposure to all kinds of carcinogens induced cancer and other diseases, against smoking and most likely also air pollution induced cancer or other diseases (truck drivers, police officers, inner city dwellers, etc.). And since cells became resistant to a variety of agents, it is speculated that there is a limitless number of experiments to be done to identify which additional agents YEL002 makes cells or animals resistant to.

It is also observed that three different cancer cell lines were not protected from radiation killing by YEL002, which indicates a use in radiation oncology or chemotherapy where the tumor can still be killed by the radiation, or chemotherapy but YEL002 protects the rest of the body from radiation or chemotherapy damage, and inhibits secondary carcinogenesis. There were no side effects besides longer life and reduction of cancer and other disease incidence and protection from exposure to radiation and many carcinogens and many other beneficial effects.

In terms of mechanism of action in a proteomics study, the chemical was capable of inducing 244 different proteins in human keratinocytes, which comprised anti-inflammatory proteins, DNA repair proteins, and 9 neuroprotective proteins. It is known that turtles at the Galapagos Islands live 150 or more years without cancer and have multiple copies of DNA repair genes and anti-inflammatory genes. The compound boosts the expression of these genes in mammals. Since it induces DNA repair and is anti-inflammatory, it may be possible to prevent and cure many diseases and extend the life.

The clinical trial on psychological, psychiatric, and memory loss endpoints shows that 10 people felt more energetic, 5 less tired, 7 less combative. 7 less argumentative, 10 less angry or aggressive. 8 experienced better quality sleep. 8 needed less sleep and 5 became nicer. It gets rid of addictions and reduces symptoms of depression and Alzheimer's disease.

Proteomics shows that YEL002 induces 9 neuroprotective functions amongst DNA repair and anti-inflammatory functions and might have a direct effect on neuronal functions. A direct effect of YEL002 on the autonomic or sympathetic nervous system, the flight or fight response, which can explain all the effects that we find.

A woman who had suffered from recalcitrant depression for 20 years was completely cured within one week and had a psychiatric profile indistinguishable from normal for the next two weeks on a PHQ-9 psychiatric test for the first time in 10 years. The effects are superior to current treatment with SSRI's and others. If they work at all, improvements take at least 4 weeks and may increase patient weight.

In this patient's assessment using a standard PHQ-9 form, the patient was assessed before and after treatment with YEL002. The PHQ-9 form assigns a numerical value corresponding to the level of depression, meaning a higher level means increased depression values. In this patient, before treatment, the values was in 3 different instances 6, 9 and 15 with designation of “very difficult” and “extremely difficult” and she cried 3 times during 30 minutes of our interview and said she hates the world. Before the test indicates mild depression to moderately severe depression. After one week on YEL002, the following two weeks, the value was 3, now with a designation of “somewhat difficult” which is the lowest in the scale and designated as minimal depression and she now cries less than once in two weeks. Since there is no lower designation this indicates no depression. This is also called snapping out of depression, which was a technical term in the past because it was not observed before now.

The same patient reported in her daily journal during snapping out of depression, Day 1: “can't focus, miserable and uncomfortable”; Day 2: “a bit lighter, not so totally depressed, 1st day out, did some errands, no anxiety” Day 3 Saturday: “ok day, no crying for a few days, just tired, no anxiety”; Day 4 Sunday: “nasty day, all alone, just hanging, bored, tired”; Day 5: “1st day out at work, hard to focus, but productivity good, 2 deals done, not stressed, no crying”; Day 6: At work, no real change, not as tired as yesterday, making lots of phone calls, struggling, but no anxiety’; Day 7: Feeling less tired, mood is good, no anxiety”; Day 8: “good sleep, feeling less tired today, feeling more hopeful, in a good mood, no anxiety”; Day 9: “good sleep, feeling better, not as tired, focus OK, no anxiety.”

Another patient had long COVID19 and diabetes and was mostly bedridden for 4 years, describing his wife as old and ugly. One week into the treatment, he did not want to stay in bed any longer, where he resumed outdoor activities and went hiking, and he described the same wife now as beautiful and loving. While he had 5-10 major arguments per day with his spouse before, after treatment it was down to 0-1.

In other observations, two alcoholics were completely cured of their alcohol addiction. They did not get completely abstinent or are against drinking. They still liked the taste of their favorite drinks, but the craving for alcohol was completely gone and they stopped drinking alone or with people who do not drink and only drink one or two glasses now in social situations with people who drink for a toast.

Another patient with Alzheimer's disease, after 4 weeks YEL002 treatment felt much better and her memory was improved.

Another person's memory was much improved. Before, once he wandered around for more than one hour to look for his car amongst thousands of others. After starting therapy, he remembers every time where he left his car even in a ten-story parking structure without any effort to specifically remember the location.

Effects on pets. My cats, Susie and Celine were the sweetest cats before I started to treat them. But after treatment they are now even sweeter, cuddlier and more attached to me, and sleep 2 hours longer per day. I call them lap cats now like the dog pendant. They lost loofiness, shyness to new visitors, quietly sit next to each other and licking each other or me and just want to sit close to me or on my lap and be petted. Before after 5 to 10 minutes of petting they just left and did something else. Now they stay for hours and the only reason for them to leave is when I stop petting them. And they are even better at play and jump exactly, landing where they intend to, don't bump into things and catch the toys the first time every time.

All publications and patent applications cited in this specification are herein incorporated by reference as if each individual publication or patent application were specifically, and individually, indicated to be incorporated by reference.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed as the best mode contemplated for carrying out this invention, but that the invention will include all embodiments falling within the scope of the appended claims.

1. A preparation comprising YEL002 as an active ingredient for the prevention or treatment of one or more psychological conditions or psychiatric diseases.

2. The preparation of claim 1, wherein the preparation is a sublingual pill.

3. The preparation of claim 1, wherein the preparation is an injection.

4. The preparation of claim 1, wherein the preparation is transdermal.

5. The preparation of claim 1, wherein the preparation is dissolved in a liquid.

6. The preparation of claim 1, wherein the preparation is dissolved in an oil.

7. The preparation of claim 6, wherein the oil is grapeseed oil.

8. The preparation of claim 1, wherein the disease or condition is one or more of:
sleep deprivation;
poor sleep quality;
anger;
aggression;
memory loss or deterioration;
addiction;
depression; and
dementia, including Alzheimer's.

9. A method of preventing or treating one or more psychological conditions or psychiatric diseases, said method comprising administering a preparation comprising YEL002 to a patient at a predetermined dosage.

10. The method of claim 9, wherein the dosage is 0.01 to 5 grams per 50 kg body weight per day.

11. The method of claim 9, wherein the dosage is 1.0 grams per 50 kg body weight per day.

12. The method of claim 9, wherein the preparation is a sublingual pill.

13. The method of claim 9, wherein the preparation is an injection.

14. The method of claim 9, wherein the preparation is transdermal.

15. The method of claim 9, wherein the preparation is YEL002 dissolved in liquid.

16. The method of claim 9, wherein the preparation is YEL002 dissolved in an oil.

17. The method of claim 16, wherein the oil is grapeseed oil.

18. The method of claim 9, wherein administering the preparation further comprises applying the oil containing YEL002 to the nasal membrane.

19. The method of claim 16, wherein administering the preparation further comprises atomizing the oil containing YEL002 and inhaling the mist.

20. The method of claim 9, wherein the disease is one or more of:
sleep deprivation;
poor sleep quality;
anger;
aggression;
memory loss or deterioration;
addiction;
depression; and
dementia including Alzheimer's.
